(19) United States
(12) Patent Application Publication
(10) Pub. No.: US 2008/0006276 A1
(43) Pub. Date: Jan. 10, 2008

(54) Face mask, in particular inhalation mask for use with a therapeutic nebuliser

(75) Inventors: Vera Kreutzmann, Seefeld (DE); Sven Rosenbeiger, Starnberg (DE); Johann Zimmermann, Bergheim (DE)

Correspondence Address: WOLF GREENFIELD & SACKS, P.C., 600 ATLANTIC AVENUE, BOSTON, MA, 02210-2206, US

(73) Assignee: Pari GmbH Spezialisten fur effektive Inhalation, Starnberg (DE)

(21) Appl. No.: 11/825,310

(22) Filed: Jul. 5, 2007

(30) Foreign Application Priority Data
Jul. 6, 2006 (DE) 10 2006 031 312.7

(51) Int. Cl. A61M 16/06; A62B 18/02

(52) U.S. Cl. 128/206.24; 128/206.28

(57) ABSTRACT

In order to improve the wearing comfort and the sealing in an upper nasal region, the present invention proposes a face mask, in particular an inhalation mask for use with a therapeutic nebuliser, comprising a main mask body that comprises a first circumferential region, which is designed for the application of the mask to a user's face, and a second region for connection to, in particular, a therapeutic nebuliser, wherein a section of the first region is designed for application to an upper nasal region of the face and comprises two sealing lips connected in series. According to the invention, the two sealing lips connected in series are formed by a first inward-facing sealing lip and a second outward-facing sealing lip.

The present invention relates to a face mask and in particular a face mask for use with a therapeutic nebuliser.

Nebulisers for therapeutic purposes, hereinafter referred to as “therapeutic nebulisers”, enable a user to inhale a medicament-containing aerosol which was produced beforehand by an aerosol generator in the therapeutic nebuliser. To inhale the aerosol, the user normally takes a mouthpiece, which is attached to the therapeutic nebuliser, in the mouth and breathes in the aerosol through the mouthpiece. However, there are groups of users for whom the use of a mouthpiece to inhale the aerosol is unsuitable or even impossible. For these groups of users, inhalation or breathing masks are used, which are placed on the user's face so that the user can inhale the aerosol of the therapeutic nebuliser which is supplied via a connecting piece.

A mask of this kind is described, for example, in the German patent DE 101 26 808 C1. The inhalation mask disclosed therein comprises a main mask body composed of a first circumferential region for the application of the mask to a user's face and a second region for connection to a therapeutic nebuliser. An inhalation mask such as the one described in German patent DE 101 26 808 C1 is conventionally made from a part made of PP or PVC. Masks of this kind in particular need to be improved with respect to the wearing comfort and the sealing function between the mask and the face.

In particular when administering the medicament-containing aerosol, it is necessary to ensure that the aerosol cannot enter the user's eyes since this could trigger severe irritation of the eyes. Consequently, with the known masks, it is necessary in particular to improve the seal between the mask and an upper nasal region. In this context, the upper nasal region should be understood as the region surrounding the upper part of the bridge of the nose and the nasal wings extending therefrom.

In addition to the aforementioned inhalation masks, also known from the prior art are so-called sleep apnoea masks. A sleep apnoea mask of this kind is described for example in U.S. Pat. No. 6,112,746. The face mask disclosed therein comprises a mask cushion for application to a user's face. This cushion also has a region designed for application in an upper nasal region of the face. This face cushion comprises two completely circumferential, inward-facing sealing lips which are connected in series in such a way that they overlap. In this context, the term “inward-facing” should be understood as follows. The region for application to the user's face is a circumferential region, defining an enclosed cross-sectional shape with an opening in its interior. “Inward facing” means here that the sealing lips point towards the opening.

A similar face mask is also known from European patent EP 0 264 772 B1. This face mask also comprises two inward-facing sealing lips connected in series in a section for application to an upper nasal region of the face, which overlap each other.

Despite these two sealing lips connected in series, the seal in the upper nasal region, in particular for use with a medicament-containing aerosol, i.e. with a therapeutic nebuliser, could be improved. Similarly, the wearing comfort could also be further improved.

Accordingly, it is the object of the present invention to further develop the face masks mentioned in the introduction in such a way that the seal between the face mask and the face in the upper nasal region and the wearing comfort are improved.

This problem is solved by a face mask having the features of claim 1. Advantageous further developments of the present invention can be seen from the dependent claims.

The present invention proposes a face mask, in particular an inhalation mask for use with a therapeutic nebuliser, comprising a main mask body. The main mask body comprises a first circumferential region, which is designed for the application of the mask to a user's face, and a second region for connection, in particular to a therapeutic nebuliser or a connecting piece of such a nebuliser. In this regard, a section of the first region, which is designed for application to an upper nasal region of the face, has two sealing lips connected in series. According to the invention, the two sealing lips connected in series are formed by a first inward-facing or inward-lying sealing lip and a second outward-facing or outward-lying sealing lip. In this regard, the terms “inward” or “outward” with reference to the above statements should be understood as meaning that the inward-facing sealing lip points inward toward the opening defined by the circumferential first region, while the second outward-facing sealing lip points away from this opening. The design of the sealing lips according to the invention enables the first region to fit well on different facial contours and in particular on the upper nasal region. The upper nasal region is in particular very different from person to person, so that this is also the most difficult region of the seal. In particular, the present invention and in particular the design of the sealing lips achieves a particularly good sealing function in this region and it is therefore possible to prevent a medicament-containing aerosol escaping and entering the eyes which can result in irritation of the eyes. In addition, the face mask according to the invention also has improved wearing comfort.

According to one embodiment of the present invention, the second sealing lip substantially extends along the entire first circumferential region. This means that the second sealing lip substantially lies over the entire circumference of the face mask, which is formed by the first circumferential region on the user's face and seals the mask against the face. In this context, the term “substantially” should be understood as meaning that the sealing lip extends over the greater part of the entire circumferential first region, but certain sub-regions can be left free in order, for example, to attach straps for the application of the mask. Advantageously, on the other hand, the first sealing lip is substantially provided only in the section for application in an upper nasal region and serves to reinforce the sealing effect in this region. In addition to the increased sealing effect, this also contributes to a further improvement of the wearing comfort.

By means of the design according to the invention of an inward-facing and an outward-facing sealing lip, the first and the second sealing lip do not overlap each other unlike the case in the aforementioned prior art.

According to an advantageous embodiment of the present invention, the second sealing lip is an outwardly curved sealing lip, i.e. it has an outwardly curved shape. This improves the close fit of the face mask to a user's face and hence the sealing effect.

On the other hand, the first sealing lip is configures as an inwardly protruding web and is advantageously membrane-shaped, i.e. thin-walled (thinner than the second sealing lip), so that it does not feel uncomfortable when the mask is applied, but without losing its sealing effect.

In order to improve the wearing comfort and the sealing effect and the suitability for connection to a connecting piece, the face mask of the present invention consists of two different materials, with a first material being used for the first region and a second material being used for the second region. This makes it possible to optimise the first region with regard to the wearing comfort and sealing effect and the second region for connection to, for example, a therapeutic nebuliser.

To this end, the first region is preferably formed from a softer or more flexible material than the second region. For example, TPE (thermoplastic elastomers) can be used for the first sealing region and PP (polypropylene) can be used for the second region for connection to a therapeutic nebuliser.

To produce a face mask of this kind in a cost-effective way, the person skilled in the art will use generally known two-component injection methods which enable two different components to be moulded in one mould in such a way that an integrally configured product can be achieved.

Further advantages, features and embodiments of the present invention are evident from the following description of an embodiment with reference to the figures, in which:

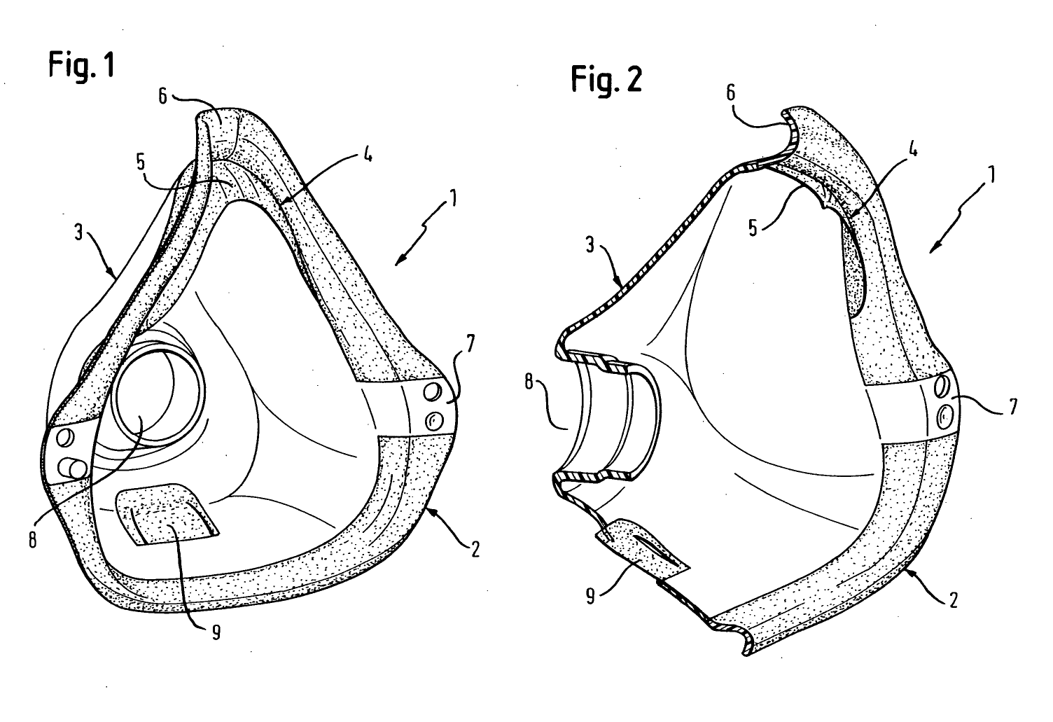

FIG. 1 shows a perspective view of a face mask according to the invention and

FIG. 2 shows a longitudinal section of the mask shown in FIG. 1, also in a perspective view.

FIG. 1 shows an inhalation mask 1 for use with a therapeutic nebuliser. The face mask comprises a main mask body consisting of a first circumferential region 2 for application of the mask to a user's face and a second region 3 for connection to a therapeutic nebuliser.

A section 4 of the first region 2 is in this regard designed for application to an upper nasal region of the face and comprises two sealing lips 5 and 6 connected in series. One of the sealing lips is configured as an inward-facing sealing lip 5 (first sealing lip) and the other is configured as an outward-facing sealing lip 6 (second sealing lip).

The outward-facing or outward-lying sealing lip 6 extends over the entire circumferential first region 2 and when in use substantially lies over the entire circumference of the sealing lip 6 on a user's face. Only in one region 7, in which the face mask is provided with straps or bands in order to fix it to the user's face, is this outward-facing sealing lip 6 much thinner, such as is evident in particular from FIG. 1. In cross-section, the outward-lying sealing lip 6 has an outwardly curved shape (see FIG. 2).

On the other hand, the inward-facing sealing lip 5 is only provided in the section for application in the upper nasal region of the face. It is configured as a thin-walled inwardly protruding web and the inward-facing edge of this sealing lip 5 approximately defines the shape of the upper nasal region. In particular, the sealing lip 5 is configured as a lamellar shape and is thinner than the sealing lip 6.

In addition, the inhalation mask shown in the figures comprises a connection 8 for connection to a connecting piece of a therapeutic nebuliser. In addition, a leaf valve 9 is provided, which allows the exhaled air to escape from the mask. During inhalation, on the other hand, it prevents outside air from flowing into the mask.

As shown in the figures, the first region 2, which is formed from the two sealing lips 5 and 6, is formed from a first material. The leaf valve 9 is also formed from this material. The second region 3, on the other hand, is formed from a different material. For example, the first region 2 can be made of TPE, while the second region 3 is made of PP. In order to improve the wearing comfort and the seal, however, it is in all cases preferable for the material of the first region 2 to be softer and more flexible or elastic than the material of the second region 3, so that the sealing lips fit well to the contour of the face. This also means that the connector 8 is optimised for connection to a therapeutic nebuliser and can be formed from a rigid material so that the therapeutic nebuliser can, on the one hand, be easily connected and, on the other, the connection to the therapeutic nebuliser can be securely and reliably maintained.

Although the present invention was explained with reference to an inhalation mask for use with a therapeutic nebuliser, the present invention can obviously also be applied to other face masks, such as, for example, sleep apnoea masks, anaesthetic masks or other face masks. It is also obvious that, in addition to the two sealing lips described in the upper nasal region, further sealing lips can be present. For example, in addition to the outward-facing sealing lip 6 and the inward-facing sealing lip 5, a further inward-lying sealing lip or a further outward-lying sealing lip can be provided. Further additional sealing lips can also be provided in other sections of the first region 2.

1. Face mask, in particular inhalation mask for use with a therapeutic nebuliser, comprising a main mask body comprising a first circumferential region, which is designed for the application of the mask to a user's face, and a second region for connection, in particular to a therapeutic nebuliser, wherein a section of said first region is designed for application in an upper nasal region of the face and comprises two sealing lips connected in series, wherein
the two sealing lips connected in series are formed by a first inward-facing sealing lip and a second outward-facing sealing lip.

2. Face mask according to claim 1, wherein the second sealing lip substantially extends along the entire circumferential first region and the first sealing lip is substantially only provided in the section for application to an upper nasal region.

3. Face mask according to claim 1, wherein the first and the second sealing lips do not overlap.

4. Face mask according to claim 1, wherein the second sealing lip has an outwardly curved shape.

5. Face mask according to claim 1, wherein the first sealing lip is configured as an inwardly protruding web.

6. Face mask according to claim 1, wherein the first region and the second region are made of different materials.

7. Face mask according to claim 6, wherein the first region is formed from a softer material than the second region.

8. Face mask according to claim 6, wherein the first and second regions are produced in the two-component injection moulding method.
